(19) United States
(12) Patent Application Publication
(10) Pub. No.: US 2004/0153006 A1
(43) Pub. Date: Aug. 5, 2004

(54) Intracorporeal devices with ionomeric polymer sleeves

(75) Inventors: Anthony C. Vrba, Maple Grove, MN (US); Horng-Ban (Howard) Lin, Maple Grove, MN (US)

Correspondence Address: David M. Crompton, CROMPTON, SEAGER & TUFTE, LLC, 331 Second Avenue South, Suite 895, Minneapolis, MN, 55401-2246, US

(73) Assignee: SciMed Life Systems, Inc.

(21) Appl. No.: 10/356,912

(22) Filed: Feb. 3, 2003

(51) Int. Cl. A61B 5/00

(52) U.S. Cl. 600/585

(57) ABSTRACT

Intracorporeal and other elongate medical devices such as guidewires, catheters and the like can be formed with an ionomeric polymer sleeve or jacket covering at least a portion of the elongate medical device to form a hydrophilic surface thereon. The ionomeric polymer sleeve or jacket can be hydrophilic and can eliminate the need for subsequent application of a traditional hydrophilic coating such as a hydrogel.

### TECHNICAL FIELD

The invention relates to medical devices and, more particularly, to intracorporeal medical devices.

### BACKGROUND

A variety of medical devices, including intracorporeal devices, employ hydrophilic coatings such as hydrogel coatings. However, hydrogel coatings can have disadvantages, including poor adherence to certain substrates, too much friction, too little permanence, and difficult methods of application. Moreover, adding a hydrogel coating requires an additional processing step. Thus, there is room for improvement in providing a hydrophilic nature to medical devices in general, and in particular, intracorporeal devices.

### SUMMARY

The invention is directed to intracorporeal and other elongate medical devices such as guidewires, catheters and the like that can be formed with an ionomeric polymer sleeve or jacket covering at least a portion thereof. This ionomeric polymer sleeve or jacket is a jacket formed of a polymer containing charged functional groups to provide a hydrophilic surface on that portion of the medical device. In preferred embodiments, the need for a separate hydrophilic coating, such as a hydrogel, is eliminated. One preferred ionomer polymer is a sulfonated polyurethane ionomer. This polymer contains negatively charged sulfonate groups and provides a hydrophilic surface.

Accordingly, an embodiment of the invention can be found in an intracorporeal device that includes an elongate body and an ionomeric polymer sleeve that is in an overlying alignment with at least a portion of the elongate body. The ionomeric polymer sleeve is preferably formed by extrusion. However, other conventional methods may by utilized.

Another embodiment of the invention can be found in an elongate medical device that includes an elongate shaft having a distal portion, an intermediate portion and a proximal portion. An ionomeric polymer jacket is preferably in coaxial alignment over at least one of the distal portion, the proximal portion or the intermediate portion of the elongate shaft to provide a hydrophilic surface.

Another embodiment of the invention can be found in a guidewire that includes an elongate core that has a distal portion, an intermediate portion and a proximal portion, and an extruded hydrophilic polymer jacket that covers at least one of the distal portion, the proximal portion or the intermediate portion of the elongate core. The ionomeric polymer jacket is preferably extruded and can eliminate the need for a separate hydrogel coating.

Another embodiment of the invention can be found in a method of forming a hydrophilic medical device that has an elongate shaft having a distal portion, an intermediate portion and a proximal portion. An ionomeric polymer jacket is positioned in coaxial alignment with the elongate shaft and is secured to the elongate shaft. The ionomeric polymer jacket is an ionomer polymer with charged functional groups. In a preferred embodiment, the ionomeric jacket is extruded over at least a portion of the shaft to provide a hydrophilic surface.

### BRIEF DESCRIPTION OF THE DRAWINGS

FIG. 1 is a perspective view of an idealized elongate medical device having an elongate shaft, with an ionomeric polymer jacket in coaxial alignment with a distal portion of the elongate shaft;

FIG. 2 is a perspective view of an idealized elongate medical device having an elongate shaft, with an ionomeric polymer jacket in coaxial alignment with an intermediate portion of the elongate shaft;

FIG. 3 is a perspective view of a portion of an idealized elongate medical device having an elongate shaft, with an ionomeric polymer jacket in coaxial alignment with a proximal portion of the elongate shaft;

FIG. 4 is a perspective view of a portion of an idealized elongate medical device having an elongate shaft, with an ionomeric polymer jacket in coaxial alignment with a portion of the elongate shaft and an intermediate polymer jacket;

FIG. 5 is a partial cross-sectional view of a portion of a guidewire, with an ionomeric polymer jacket in coaxial alignment with a portion of the guidewire;

FIG. 6 is a partial cross-sectional view of a portion of another guidewire, with an ionomeric polymer jacket in coaxial alignment with a portion of the guidewire;

FIG. 7 is a plan view of a catheter;

FIG. 8 is a partial cross-sectional side view of one embodiment of the catheter of FIG. 7; and

FIG. 9 is a partial cross-sectional view of another embodiment of the catheter of FIG. 7.

### DETAILED DESCRIPTION

The present invention describes intracorporeal devices that include an ionomeric polymer sleeve. An intracorporeal device can include any device that can be placed temporarily or permanently within the body. The ionomeric polymer sleeve preferably attracts or binds water and thus can pass more easily through an aqueous environment such as that found within the human body.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. Thus, for example, reference to a composition containing “a compound” includes a mixture of two or more compounds. As used in this specification and the appended claims, the term “or” is generally employed in its sense including “and/or” unless the content clearly dictates otherwise.

A hydrophilic polymer is a polymer that attracts or binds water molecules when the polymer is placed in contact with an aqueous system. Examples of aqueous systems that can provide water molecules that can bind to a hydrophilic polymer include blood and other bodily fluids. When a hydrophilic polymer comes into contact with such a system, water molecules can bind to the polymer via mechanisms such as hydrogen bonding between the water molecules and substituents or functional groups present within or on the polymer.

One class of polymers that can be considered as hydrophilic includes ionomer polymers. An ionomer polymer is a polymer that can be considered as containing covalent bonds between elements within a chain while containing ionic bonds between chains. An ionomer polymer is a polymer that has charged functional groups appended to the polymer chain. The charged functional groups can be positively charged, in which case the polymer can be referred to be a cationomer, or the functional groups can be negatively charged, in which case the polymer can be referred to as an anionomer.

An ionomeric polymer can be formed using a variety of negatively charged functional groups. The negatively charged functional group can be added to a previously formed polymer, or the negatively charged functional groups can be part of one or more of the monomers used to form the ionomeric polymer.

Examples of suitable negatively charged functional groups include sulfonates and carboxylates. The ionomeric polymer can, in particular, include sulfonate functional groups. These groups are negatively charged and can readily hydrogen bond sufficient amounts of water when brought into contact with a source of water such as an aqueous system.

As represented generically in the Figures, an intracorporeal device 10 can include elongate medical devices such as catheters, including balloon catheters, stent catheters, guide catheters, urinary catheters, and biliary catheters, introducer sheaths, and guidewires. An intracorporeal device 10 can also include biliary equipment such as endoscopes and various intravascular devices such as distal protection devices. Other devices include needles, wound drains and shunts. Intracorporeal devices 10 can be considered as including elongate bodies or shafts upon which a polymer sleeve such as a hydrophilic polymer sleeve can be applied.

Intracorporeal devices 10 such as those described herein can be formed from a variety of different substrates, depending on the end use of the device 10. Substrates that can be used include both metallic and non-metallic materials. Examples of possible metallic materials include stainless steel, tantalum, gold, titanium, and nickel-titanium alloy.

Examples of possible non-metallic materials include but are not limited to poly(L-lactide) (PLLA), poly(D,L-lactide) (PLA), polyglycolide (PGA), poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), polyethylene oxide (PEO), polydioxanone (PDS), polycaprolactone (PCL), polyhydroxylbutyrate (PHBT), poly(phosphazene), poly D,L-lactide-co-caprolactone) (PLA/PCL), poly(glycolide-co-caprolactone) (PGA/PCL), polyanhydrides (PAN), poly(ortho esters), poly(phosphate ester), poly(amino acid), poly(hydroxy butyrate), polyacrylate, polyacrylamid, poly(hydroxyethyl methacrylate), polyurethane, polysiloxane and their copolymers.

FIGS. 1 through 4 represent a portion of an intracorporeal device 10 that, as indicated above, can represent a variety of different elongate medical devices. Exemplary elongate medical devices include guidewires and catheters, and thus the intracorporeal device 10 is illustrated as being cylindrical. FIG. 1 represents a distal portion of the intracorporeal device 10. An ionomeric polymer sleeve is illustrated in coaxial alignment with the distal end of the intracorporeal device 10.

As illustrated in FIGS. 1 through 4, the intracorporeal device 10 has a solid elongate shaft 18. If, however, the intracorporeal device 10 represents a catheter such as a guide catheter or a balloon catheter or an introducer sheath (not specifically illustrated), the elongate shaft 18 can be hollow, and can include one or more lumens extending through the elongate shaft 18. The elongate shaft 18 may or may not be cylindrical in shape, depending on the type or configuration of any lumens that extend through the elongate shaft. Particular guidewires can also be hollow.

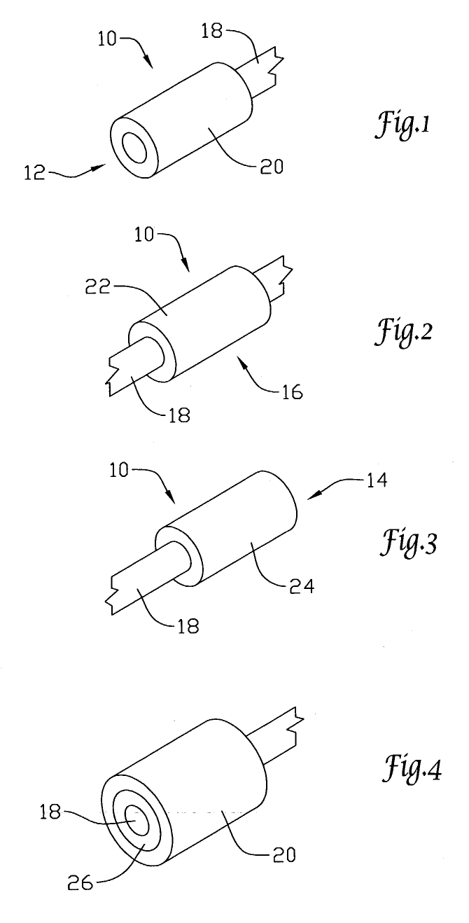

FIGS. 2 and 3 show ionomeric polymer sleeves 22, 24 that are in coaxial alignment with an intermediate portion 16 of an elongate shaft 18 and a proximal portion 14 of an elongate shaft 18, respectively. An ionomeric polymer sleeve can be placed in coaxial alignment with any portion of the elongate shaft 18. An ionomeric polymer sleeve can also be placed in coaxial alignment with the entire elongate shaft 18. This can be illustrated by contemplating, in combination, the distal polymer sleeve 20 of FIG. 1, the intermediate polymer sleeve 22 of FIG. 2, and the proximal polymer sleeve 24 of FIG. 3.

In particular, the ionomeric polymer sleeve can form one or more of a distal sleeve 20 intended for coaxial alignment with the distal portion 12 of an elongate shaft 18, an intermediate sleeve 22 intended for coaxial alignment with the intermediate portion 16 of an elongate shaft 18, or a proximal sleeve 24 intended for coaxial alignment with the proximal portion 14 of an elongate shaft 18.

As illustrated for example in FIG. 4, an elongate shaft 18 can include an ionomeric polymer sleeve 20 that is in coaxial alignment with the elongate shaft 18 and with an intermediate polymer sleeve 26. The intermediate polymer sleeve 26 can be hydrophilic, or the intervening polymer sleeve 26 can be a non-hydrophilic polymer sleeve such as a non-sulfonated polyurethane. In preferred embodiments, the elongate medical device 10 has an elongate shaft 18 having a distal portion 12, a proximal portion 14 and an intermediate portion 16 that is positioned between the distal and proximal portions 12, 14. An extruded hydrophilic polymer jacket is provided and is positioned in coaxial alignment with the elongate shaft 18. The ionomeric polymer jacket is preferably extruded and secured to the elongate shaft 18. The polymer sleeve can be co-extruded directly onto the intracorporeal device 10 or onto an elongate shaft 18 of the intracorporeal device 10. The polymer sleeve can be extruded as a tube that can subsequently be placed onto the intracorporeal device 10. The polymer sleeve can be co-extruded with the elongate shaft 18 of the intracorporeal device 10 and an intermediate polymer jacket 26 that may or may not be hydrophilic. The intermediate polymer jacket 26 can be a nonionic polymer sleeve such as a traditional polyurethane sleeve.

The polymer sleeve can be extruded having an inner diameter that is greater than an outer diameter of the intracorporeal device 10, in which case the intracorporeal device 10 can be positioned within the polymer sleeve and the polymer sleeve can be reduced in diameter by application of heat or by being placed through an appropriately sized die.

The polymer sleeve can be extruded having an inner diameter that is equal to or even smaller than an outer diameter of the intracorporeal device 10, in which case the polymer sleeve can be pressurized or otherwise inflated prior to insertion of the intracorporeal device 10. Subsequently deflating the polymer sleeve so that it regains its inner diameter can secure the polymer sleeve in place on the intracorporeal device 10.

As illustrated in the Figures, the polymer sleeve can be extruded in multiple sections 20, 22, 24. One section 20 can be intended, for example, for placement on a distal portion 12 of an intracorporeal device 10 while a second section 22 or 24 can be intended for placement on a proximal or intermediate portion 14, 16 of the intracorporeal device 10.

For example, the polymer sleeve can form one or more of a distal sleeve 20 intended for coaxial alignment with the distal portion 12 of an elongate shaft 18, an intermediate sleeve 22 intended for coaxial alignment with the intermediate portion 16 of an elongate shaft 18, or a proximal sleeve 24 intended for coaxial alignment with the proximal portion 14 of an elongate shaft 18.

Each section 20, 22 or 24 can, for example, be formed from a polyurethane having the same level of functional group substitution. There may be processing advantages to applying the polymer sleeve to the intracorporeal device 10 in sections 20, 22, 24, even if each section 20, 22, 24 is chemically identical.

Alternatively, each section 20, 22, 24 of the polymer sleeve can be formed from a polyurethane having, for each section 20, 22, 24, a different level of functional group substitution. It may be desirable, for example, to envelop the distal end 12 of an intracorporeal device 10 with a highly hydrophilic polymer sleeve, while the proximal and/or intermediate portions 14, 16 of the intracorporeal device 10 have a polymer sleeve section 22, 24 that is less hydrophilic than the distal portion.

In particular, it can be advantageous to have a distal jacket 20 that is extruded from an ionomeric polymer having a first degree of charged functional group substitution, an intermediate jacket 22 that is extruded from an ionomeric polymer having a second degree of charged functional group substitution and a proximal jacket 24 that is extruded from an ionomeric polymer having a third level of charged functional group substitution. The first degree of charged functional group substitution can be greater than the second degree of charged functional group substitution, which itself can be greater than the third degree of charged functional group substitution.

After the hydrophilic sleeve has been positioned and secured on an elongate shaft 18 of an intracorporeal device 10, it can be advantageous to perform various post-processing steps on the intracorporeal device 10. For example, if part or all of the elongate shaft 18 and hydrophilic polymer sleeve have an overall diameter that is greater than desired, the intracorporeal device 10 can, as discussed above, be fed through a heated die that can adjust the overall diameter of the intracorporeal device 10.

For some applications, it can be useful for a distal end 12 of the intracorporeal device 10 to have a particular coefficient of friction, while perhaps an intermediate portion 16 or a proximal portion 14 has a higher coefficient of friction. This can be useful if the intermediate or proximal portions 16 or 14 of the device 10 are intended to be manually handled by a physician or other professional during the use, as the higher coefficient of friction can aid in gripping the device 10. Differing coefficients of friction can be achieved by grinding or sanding a portion of the intracorporeal device 10.

For particular applications, it can be advantageous for differing portions of the intracorporeal device 10 to have different geometries. For example, a portion of the intracorporeal device 10 can have a circular cross-section, while other portions have a non-circular cross-section. Varying cross-sectional geometries can be achieved by removing material, such as by grinding, or by the application of heat and pressure.

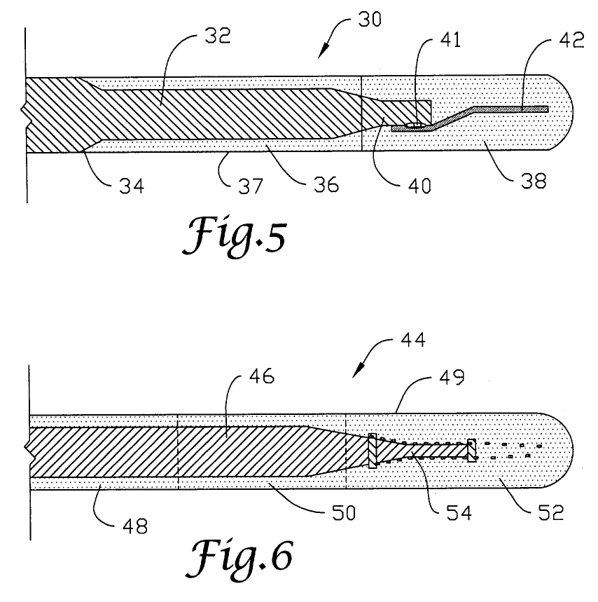

Guidewires represent an exemplary application of the present invention and thus will be discussed as an illustrative but non-limiting example. FIG. 5 shows a guidewire distal portion 30 that can have a solid cross-section or a hollow cross-section, and may be formed of any materials suitable for use, dependent upon the desired properties of the guidewire. Some examples of suitable materials include metals, metal alloys, and polymers. The guidewire distal portion 30 can be formed of a relatively flexible material such as a straightened superelastic or linear elastic alloy (e.g., nickel-titanium) wire, or alternatively, a polymer material, such as a high performance polymer. Alternatively, a metal or metal alloy such as stainless steel, nickel-chromium alloy, nickel-chromium-iron alloy, cobalt alloy, or other suitable material can be used.

The guidewire distal portion 30 includes a core wire 32. As illustrated, the core wire 32 includes two tapered regions and two constant diameter regions such that the core wire 32 has a geometry that decreases in cross-sectional area toward the distal end 40 thereof. In some embodiments, these tapers and constant diameter regions can be adapted and configured to obtain a transition in stiffness and provide a desired flexibility characteristic.

A wire or ribbon 42 is attached adjacent the distal end 40 of the core wire 32. The wire or ribbon 42 can be a fabricated or formed wire structure. As shown, the ribbon 42 is a generally straight wire that overlaps with and is attached to the core wire 32 at an attachment point 41.

The ribbon 42 can be made of any suitable material and sized appropriately to give the desired characteristics, such as strength and flexibility characteristics. Some examples of suitable materials include metals, metal alloys, polymers, and the like. The ribbon 42 can be formed of a metal or metal alloy such as stainless steel, nickel-chromium alloy, nickel-chromium-iron alloy, cobalt alloy, a nickel-titanium alloy, such as a straightened super elastic or linear elastic alloy (e.g., nickel-titanium) wire. The ribbon or wire 42 can function as a shaping structure or a safety structure.

FIG. 6 illustrates a guidewire distal portion 44 that includes a coiled safety and/or shaping structure 54 that is disposed about a portion of the core wire 46. As shown, the coiled structure 54 is a coiled ribbon that overlaps with or surrounds a portion of the distal-most tapered portion of the core wire 46.

The guidewire distal portions 30 and 44 also include a polymer sleeve 37 that covers at least a portion of the core wires 32 and 46, respectively. First, with respect to FIG. 5, the polymer sleeve 37 is illustrated as having a proximal polymer sleeve 36 and a distal polymer sleeve 38. As discussed with respect to the intracorporeal device 10, each of the first polymer sleeve 36 and the second polymer sleeve 38 can be formed of an ionomeric polymer bearing charged functional groups.

A sulfonated polyurethane is an exemplary material that can be used to form the proximal polymer sleeve 36 and/or the distal polymer sleeve 38. Each sleeve 36, 38 can include polyurethane having identical levels of sulfonation. Alternatively, the proximal polymer sleeve 36 can, for example, have a level of sulfonate substitution that is either greater than or less than a level of sulfonate substitution in the distal polymer sleeve 38.

As illustrated, the proximal polymer sleeve 36 and the distal polymer sleeve 38 have a constant, identical outer diameter. In other embodiments, part or all of the proximal polymer sleeve 36 and the distal polymer sleeve 38 can be subjected to a post-forming processing step, such as grinding, in order to impart a different external geometry to the guidewire. In FIG. 5, the proximal polymer sleeve 36 has an external diameter that is equal to that of the core wire 32 at point 34.

FIG. 6, however, illustrates a core wire 46 that lacks a narrowing point such as the point 34 of FIG. 5. In this embodiment, the polymer sleeve 49 actually includes a proximal polymer sleeve 48, an intermediate polymer sleeve 50 and a distal polymer sleeve 52. Each of the sleeves 48, 50 and 52 can include polyurethane having identical levels of sulfonation. Alternatively, each polymer sleeve can, for example, have a level of sulfonate substitution that is either greater than or less than a level of sulfonate substitution in the other polymer sleeves.

As illustrated, the proximal polymer sleeve 48, the intermediate polymer sleeve 50 and the distal polymer sleeve 52 have a constant, identical outer diameter. In other embodiments, part or all of the polymer sleeve 49 can be subjected to a post-forming processing step, such as grinding, in order to impart a different external geometry to the guidewire.

The polymer sleeve 37, 49 can be disposed around and attached to the guidewire distal portion 32, 46 using any suitable technique for the particular material used. In some embodiments, the polymer sleeve 37, 49 can be attached by heating a sleeve of polymer material to a temperature until it is reformed around the distal guidewire portion 30, 44. The polymer sleeve 37, 49 can be attached using heat shrinking techniques. The polymer sleeve 37, 49 may be finished, for example, by a centerless grinding or other method to provide the desired diameter and to provide a smooth outer surface.

In some embodiments, the polymer sleeve 37, 49, or portions thereof, can include, or be doped with, radiopaque material to make the polymer sleeve 37, 49, or portions thereof, more visible when using certain imaging techniques, for example, fluoroscopy techniques. Any suitable radiopaque material known in the art can be used. Some examples include precious metals, tungsten, barium subcarbonate powder, and the like, and mixtures thereof.

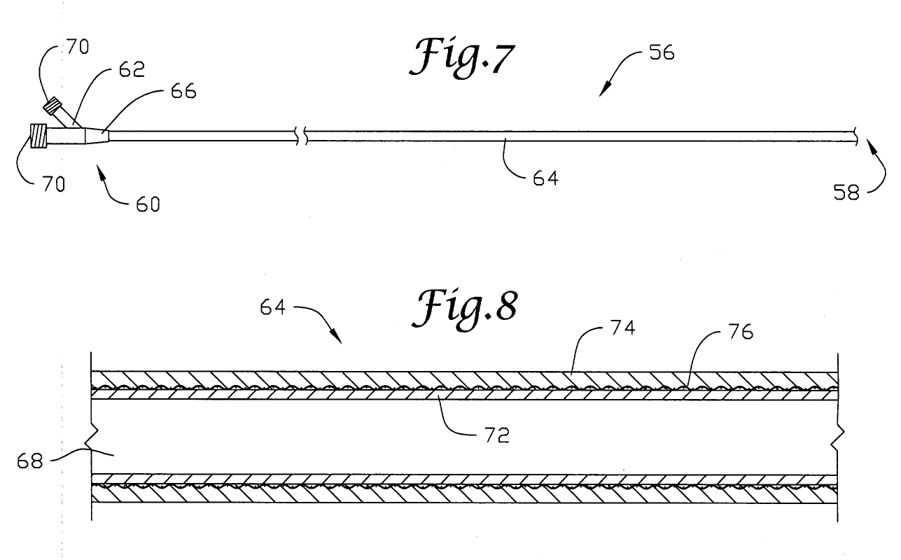

Catheters represent another exemplary application of the present invention and thus will be discussed as an illustrative but non-limiting example. FIG. 7 shows a sectional side view of a catheter 56 that has a proximal end 60 and a distal end 58. A manifold 62 is positioned at the proximal end 60 and is connected to a catheter shaft 64 and includes a strain relief 66. The manifold 62 generally contains ports 70 that allow for fluid-tight connections. A luer-lock fitting is an example of a fluid-tight fitting attached to the manifold ports 70.

The distal end 58 of the catheter 56 can be arranged and configured depending on the intended use for the catheter 56. For example, if the catheter 56 is intended for use as a guide catheter, the distal end 58 will include a soft tip (not illustrated) made of a soft material that minimizes trauma to the surrounding tissue as catheter 56 is advanced to, and ultimately engaged with, its final destination within the vasculature. Alternatively, if the catheter 56 is a balloon catheter, the distal end 58 preferably will include the appropriate structure.

FIG. 8 is a partial cross-sectional side view of the catheter 56 that illustrates particular structural features forming at least a portion of a preferred catheter shaft 64. The catheter shaft 64 includes an inner polymer layer member 72 that is surrounded by a support member layer 76. An outer tubular member 74 subsequently surrounds the support member layer 76.

The inner polymer layer member 72 is formed of a polymer using an appropriate method. For example, the inner polymer layer member 72 can include a polymer, for example polytetrafluoroethylene, that is coated onto a mandrel and appropriately cured to provide a lubricious lumen wall.

FIG. 8 further illustrates support member layer 76 applied over inner polymer layer member 72. An appropriate support material generally known can be used. In some embodiments, support member layer 76 can include a single braided filament or can include two or more interwoven braided filaments that extend over at least a portion of the length of catheter shaft 64.

The support member layer 76 can be prefabricated and then disposed over the inner polymer layer member 72, or can be constructed directly onto the inner polymer layer member 72. In embodiments where the support member layer 76 is constructed over the inner polymer layer member 72, the filaments may be wrapped around inner polymer layer member 72 at a tension such that the filaments embed slightly into the inner polymer layer member 72. A further process for partially embedding the support member layer 76 into the inner polymer layer member 72 involves heat. In this process, the newly braided catheter is passed through a heated dye that allows the filaments to partially embed into inner polymer layer member 72 without significantly altering the polymeric structure of inner polymer layer member 72.

Outer tubular member 74 is subsequently put onto or formed over support member layer 76. Outer tubular member 74 is generally formed of polymer material applied around the support member layer 76 and inner polymer layer member 72. Any appropriate method of applying the outer tubular member can be used. For example, in some embodiments, the catheter shaft 64 (including the support member layer 76 and inner polymer layer member 72) is passed through an extruder which applies a polymer that flows into the interstitial spaces of support member layer 76 and forms a tubular outer layer 74.

The outer tubular member 74, in preferred embodiments, is formed from an ionomeric polymer that has substituted charged functional groups. A sulfonated polyurethane is an exemplary polymer that has been substituted with charged functional groups. The outer tubular member 74 can be extruded as a single piece, from a sulfonated polyurethane having a particular level of sulfonate substitution.

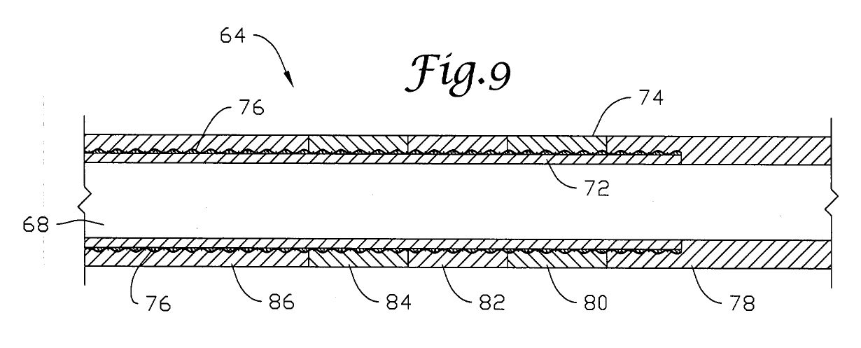

FIG. 9 is a partial cross-sectional view of an alternative design for the catheter 56 illustrating particular features of the catheter shaft 64. It can be seen that the outer tubular member 74 extends distally past the end of the inner layer 72 and the support member layer 76. More importantly, the outer tubular member 74 is illustrated as being formed from a plurality of segments 78, 80, 82, 84 and 86. Each segment 78, 80, 82, 84 and 86 can be formed from an ionomeric polymer such as a sulfonated polyurethane. Each segment 78, 80, 82, 84 and 86 can be formed from a sulfonated polyurethane having the same level of sulfonate substitution. Each segment 78, 80, 82, 84 and 86 can be formed from a sulfonated polyurethane that has a level of sulfonate substitution that is either greater than or less than the level of sulfonate substitution of any of the other segments 78, 80, 82, 84 and 86. Depending on the intended use, some segments can be formed of non-ionomeric polymers as known in the art.

The portion of catheter shaft 64 illustrated in FIG. 9 includes an outer tubular member 74 having five distinct sections. Depending on the intended use of the catheter 56, the outer tubular member 74 can be formed from more than five sections, or can be formed from less than five sections. The outer tubular member 74 can be formed from a single section, or can be formed from two or three or four sections.

The polymers used within the context of the present invention can include any known polymers having charged functional groups which form an ionomeric polymer. In some embodiments, polyurethanes bearing positively or negatively charged functional groups can be used. In particular embodiments, the polymer is a polyurethane substituted with negatively charged functional groups. In particular, the ionomeric polymer can be a sulfonated polyurethane or a carboxylated polyurethane. A sulfonated polyurethane can be a polyurethane that is substituted with alkyl sulfonate groups and, in particular, can be substituted with propyl sulfonate groups. The ionomeric polymer can also be a copolymer of sulfonated polyurethane and non-sulfonated polyurethane.

A polyurethane can be formed from monomers, chain extenders or oligomers that include a desired functional group that can provide a polymer with desired anionomer character. In some embodiments, a diamine disulfonic acid can be used as a chain extender in synthesizing a sulfonated polyurethane. In particular, a sulfonated polyurethane can be produced using 4,4′-diamino-2,2′-biphenyl disulfonic acid as a chain extender. Alternatively, a polyurethane can be formed, and desired functional groups such as sulfonate groups can subsequently be added via a grafting reaction.

An illustrative but non-limiting method of forming a sulfonated polyurethane is described herein. A polyurethane can be formed by first reacting a diisocyanate with an active hydrogen source to create a polyurethane backbone, and subsequently substituting a desired functional group. For example, a desirable functional group includes a sulfonate functional group. A sulfonate functional group can be added to a polyurethane backbone by reacting the polyurethane with a molecule bearing the desired substituent. An example of a desired substituent is a pendent propyl sulfonate group.

One way of adding this functional group is to react the polyurethane backbone with propane sulftone, which is also known as 1,2-oxathiolane-2,2-dione and has the following structure:

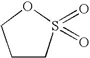

Polyurethanes suitable for use in the present invention can also include copolymers formed by reacting a diisocyanate, a diol and an ether. In particular, a suitable polyurethane can be formed by reacting methylene bis-(p-phenyl isocyanate) (MDI), N-methyldiethanolamine (MDEA) and poly(tetra-methylene oxide) (PTMO). Alternatively, 1,4-butanediol can be used as a chain extender in place of the MDEA.

A carboxylated polyurethane can be formed in a variety of ways. An illustrative but non-limiting method is described herein. A polyurethane bearing pendent carboxyl groups can be formed by reacting an aliphatic diisocyanate, a diol component and a carboxylic acid. In particular, a carboxylated polyurethane polymer can be produced as a reaction product of a diol component, an aliphatic diisocyanate, water and a 2,2-di-(hydroxymethyl) alkanoic acid. Alternatively, an amount of amine, such as diglycolamine can be used for at least a portion of the water in the reaction to form the reaction product.

The diol component can include a polyoxyalkylene diol, such as polyoxyethylene diol having a molecular weight of from about 400 to about 20,000, polyoxypropylene diol having a number average molecular weight of about 200 to about 2,500, block copolymers of ethylene oxide and propylene oxide having a molecular weight of about 1,000 to about 9,000 and polyoxytetramethylene diol having a number average molecular weight of about 200 to about 4,000.

The polyurethane can include a low molecular weight alkylene glycol such as ethylene glycol, propylene glycol, 2-ethyl-1-1,3-hexanediol, tripropylene glycol, triethylene glycol, 2,-4-pentane diol, 2-methyl-1,3-propanediol, 2,-methyl-1,3-pentanediol, cyclohexanediol, cyclohexanedimethanol, dipropylene glycol, diethylene glycol, and mixtures thereof.

An amine can be used in the reaction for at least a portion of the water in the reaction mixture. The amine can be diglycolamine, although other amines such as ethylene diamine, propylene diamine, monoethanolamine, diglycolamine, and propylene diamine can also be used.

The diisocyanate used can include both aliphatic and aromatic types and mixtures thereof. An example of a suitable isocyanate is methylene bis(cyclohexyl-4-isocyanate). Other examples of diisocyanates are trimethyl hexamethylene diisocyanate and isophorone diisocyanate. Representative examples of aliphatic diisocyanates include tetramethylene diisocyanate, hexamethylene diisocyanate, trimethylene diisocyanate, trimethylene hexamethylene diisocyanate, cyclohexyl 1,2-diisocyanate, cyclohexylene 1,4-diisocyanate, and aromatic diisocyanates such as 2,4-toluene diisocyanates and 2,6-toluene diisocyanates.

We claim:

1. An intracorporeal device comprising:
an elongate body; and
an ionomeric polymer sleeve overlying at least a portion of the elongate body to form a hydrophilic surface thereon.

2. The intracorporeal device of claim 1, wherein the intracorporeal device is selected from the group consisting of a catheter, a guidewire, an introducer sheath or a distal protection device.

3. The intracorporeal device of claim 1, wherein the ionomeric polymer sleeve comprises a polymeric material that has been substituted with charged functional groups.

4. The intracorporeal device of claim 1, wherein the ionomeric polymer sleeve comprises an extruded ionomeric polymer.

5. The intracorporeal device of claim 1, wherein the ionomeric polymer sleeve comprises sulfonated polyurethane.

6. An elongate medical device comprising:
an elongate shaft having a distal portion, a proximal portion and an intermediate portion therebetween; and
an ionomeric polymer jacket coaxially overlying at least one of the distal portion, the proximal portion or the intermediate portion of the elongate shaft.

7. The elongate medical device of claim 6, wherein the elongate medical device is selected from the group consisting of a catheter, a guidewire or an introducer sheath.

8. The elongate medical device of claim 6, wherein the ionomeric polymer jacket comprises one or more of a distal jacket coaxially overlying the distal portion of the elongate shaft, an intermediate jacket coaxially overlying the intermediate portion of the elongate shaft, and a proximal jacket coaxially overlying the proximal portion of the elongate shaft.

9. The elongate medical device of claim 6, wherein the ionomeric polymer jacket comprises, in combination, the distal jacket, the intermediate jacket and the proximal jacket, and each of the distal jacket, the intermediate jacket and the proximal jacket comprises an ionomeric polymer having charged functional groups.

10. The elongate medical device of claim 9, wherein the distal jacket is extruded from an ionomeric polymer having a first degree of charged functional group substitution, the intermediate jacket is extruded from an ionomeric polymer having a second degree of charged functional group substitution and the proximal jacket is extruded from an ionomeric polymer having a third level of charged functional group substitution.

11. The elongate medical device of claim 10, wherein the first degree of charged functional group substitution, the second degree of charged functional group substitution and the third degree of charged functional group substitution are substantially equal.

12. The elongate medical device of claim 10, wherein the first degree of charged functional group substitution is greater than the second degree of charged functional group substitution, which itself is greater than the third degree of charged functional group substitution.

13. The elongate medical device of claim 9, wherein the ionomeric polymer comprises a polyurethane substituted with negatively charged functional groups.

14. The elongate medical device of claim 9, wherein the ionomeric polymer jacket comprises a sulfonated polyurethane or a carboxylated polyurethane.

15. The elongate medical device of claim 14, wherein the ionomeric polymer jacket comprises a polyurethane substituted with alkyl sulfonate functional groups.

16. The elongate medical device of claim 9, wherein the ionomeric polymer jacket comprises a urethane-sulfonated urethane copolymer.

17. The elongate medical device of claim 6, wherein an intermediate extruded polymer jacket is positioned between the elongate shaft and the ionomeric polymer jacket.

18. A guidewire comprising:
an elongate core having a distal portion, a proximal portion and an intermediate portion therebetween; and
an ionomeric polymer jacket covering at least one of the distal portion, the proximal portion or the intermediate portion of the elongate core.

19. The guidewire of claim 18, wherein the elongate core comprises a stainless steel core wire or a NiTi core wire.

20. The guidewire of claim 18, wherein the ionomeric polymer jacket comprises one or more of a distal jacket coaxially overlying the distal portion of the elongate core, an intermediate jacket coaxially overlying the intermediate portion of the elongate core, and a proximal jacket coaxially overlying the proximal portion of the elongate core.

21. The guidewire of claim 18, wherein the ionomeric polymer jacket comprises, in combination, the distal jacket, the intermediate jacket and the proximal jacket, and each of the distal jacket, the intermediate jacket and the proximal jacket comprises an ionomeric polymer having substituted charged functional groups.

22. The guidewire of claim 21, wherein the distal jacket is extruded from an ionomeric polymer having a first degree of charged functional group substitution, the intermediate jacket is extruded from an ionomeric polymer having a second degree of charged functional group substitution and the proximal jacket is extruded from an ionomeric polymer having a third level of charged functional group substitution.

23. The guidewire of claim 22, wherein the first degree of charged functional group substitution, the second degree of charged functional group substitution and the third degree of charged functional group substitution are substantially equal.

24. The guidewire of claim 22, wherein the first degree of charged functional group substitution is greater than the second degree of charged functional group substitution which itself is greater than the third degree of charged functional group substitution.

25. The guidewire of claim 18, wherein the ionomeric polymer jacket comprises a polyurethane substituted with negatively charged functional groups.

26. The guidewire of claim 25, wherein the ionomeric polymer jacket comprises a sulfonated polyurethane or a carboxylated polyurethane.

27. The guidewire of claim 26, wherein the ionomeric polymer jacket comprises a polyurethane substituted with alkyl sulfonate functional groups.

28. The guidewire of claim 18, wherein the ionomeric polymer jacket comprises a urethane-sulfonated urethane copolymer.

29. The guidewire of claim 18, wherein the ionomeric polymer jacket comprises an inner polymer sleeve in coaxial alignment with an outer ionomeric polymer sleeve.

30. A method of forming a hydrophilic medical device, comprising the steps of:
providing an elongate shaft having a distal portion, a proximal portion and an intermediate portion therebetween;
providing an ionomeric polymer jacket which includes an ionomeric polymer with charged functional groups;
positioning the ionomeric polymer jacket in coaxial alignment with the elongate shaft; and
securing the ionomeric polymer jacket to the elongate shaft.

31. The method of claim 30, wherein the ionomeric polymer jacket is extruded directly onto the elongate shaft.

32. The method of claim 30, wherein the ionomeric polymer jacket is co-extruded with the elongate shaft and an intermediate polymer jacket therebetween.

33. The method of claim 30, wherein the ionomeric polymer jacket is first extruded, and then is subsequently placed onto the elongate shaft.

34. The method of claim 33, wherein the ionomeric polymer jacket is placed over the elongate shaft to form an assembly that is subsequently contacted with a heated die to secure the ionomeric polymer jacket on the elongate shaft.

35. The method of claim 30, wherein the ionomeric polymer jacket comprises a polyurethane with alkyl sulfonate functional groups.
